(19) United States
(12) Patent Application Publication
(10) Pub. No.: US 2015/0086518 A1
(43) Pub. Date: Mar. 26, 2015

(54) METHODS FOR GENERATING PANCREATIC TISSUE

(71) Applicant: The General Hospital Corporation, Boston, MA (US)

(72) Inventors: Harald C. Ott, Boston, MA (US); Claudius Conrad, Boston, MA (US)

(21) Appl. No.: 14/559,401

(22) Filed: Dec. 3, 2014

Related U.S. Application Data
(63) Continuation of application No. 13/504,358, filed on Dec. 19, 2012, now abandoned.
(60) Provisional application No. 61/256,247, filed on Oct. 29, 2009.

(51) Int. Cl. A61K 35/39; C12N 5/071

(52) U.S. Cl. CPC A61K 35/39; C12N 5/0677; C12N 2502/1352; C12N 2502/11; C12N 2502/22
USPC 424/93.7; 435/373; 435/1.1

(57) ABSTRACT

This document provides methods and materials related to tissue generation. For example, methods for generating pancreatic tissue and providing a population of hormone-secreting cells, e.g., insulin-producing cells in a human subject are provided.

### CLAIM OF PRIORITY

This application is a continuation of U.S. patent application Ser. No. 13/504,358, filed on Dec. 19, 2012, which is the national stage of International Patent Application No. PCT/US2010/054689, filed on Oct. 29, 2010, and claims the benefit of U.S. Provisional Patent Application Ser. No. 61/256,247, filed on Oct. 29, 2009. The entire contents of the foregoing are hereby incorporated by reference.

### TECHNICAL FIELD

This document provides methods and materials related to tissue generation. For example, this document provides methods for generating pancreatic tissue and providing a population of insulin-producing cells in a human subject.

### BACKGROUND

Type I diabetes is an autoimmune disease characterized by the destruction of insulin-producing pancreatic cells (islet beta cells) and, as a consequence, lack of insulin production. Exhaustion of islet cell function in type II diabetes leads to insulin dependence. The standard therapeutic regimen for insulin-dependent diabetes is exogenous insulin replacement therapy to maintain glucose homeostasis. Pancreas transplantation is the only treatment for Type I diabetes and insulin-dependent Type II diabetes (referred to collectively herein as “insulin-dependent diabetes”) that can consistently establish insulin independence. Whole pancreas transplantation is technically challenging and is associated with significant post-transplant morbidity. The scarcity of transplantable donor organs and the need for immunosuppressive therapy also limit the number and outcomes of pancreas transplantation.

An alternative to whole organ transplants is the transplantation of isolated, allogeneic insulin-producing cells into the liver via an injection into the portal vein. Despite advances in the field, however, allogeneic islet cell transplantation methods are typically inadequate to establish insulin independence in human patients due to low engraftment rates and the inability of transplanted islet cells to maintain long-term insulin production. In addition, current culture methods cannot sustain human islet cells in culture for longer than a few days, and have thus found only limited clinical use.

### SUMMARY

This document provides methods and materials that can be used to generate tissue. For example, the methods and materials provided herein can be used to promote the generation of functional pancreatic tissue. As described herein, this document provides methods and materials for generating a composite tissue matrix seeded with cells, e.g., hormone-producing cells, endothelial cells, stem cells, and other supportive cells. This document also provides methods and materials for using such a composition for regenerating pancreatic tissue. As described herein, this document provides, for example, methods and materials by which clinicians and other professionals can contact a stem cell-seeded tissue matrix at the site of surgical repair in order to promote regenerating pancreatic tissue and promoting insulin production following transplantation. Such treatment methods can have substantial value for clinical use.

In one aspect, this document features a method for generating a functional bioartificial pancreatic tissue. The method can comprise providing a decellularized pancreatic tissue matrix; directly seeding the decellularized pancreatic tissue matrix with one or both of a hormone secreting cell (e.g., an islet cell, e.g., an alpha, beta, delta, PP, or epsilon cell), e.g., cells that secrete hormones of the Islet of Langerhans such as insulin, glucagon, pancreatic polypeptide, amylin, ghrelin, and somatostatin) and a regenerative cell; seeding the matrix with an endothelial cell by vascular perfusion; and maintaining the seeded matrix under conditions and for a time sufficient for tissue growth to occur. In some embodiments, the hormone secreting cells are obtained by differentiating a regenerative cell, either before or after seeding into the matrix. In some embodiments, the islet cells are in whole or partially intact islets of Langerhans. The method can thereby generate functional pancreatic tissue. The method can further comprise assaying for insulin secretion from the tissue. The method can provide a decellularized pancreatic tissue matrix comprises obtaining tissue comprising a pancreas or a portion thereof, and decellularizing the pancreas under conditions such that the acellular tissue matrix, including the vasculature, substantially retains morphology of an extracellular matrix of the pancreatic tissue prior to decellularization. The regenerative cell can be, e.g., a mesenchymal stem cell, an autologous stem cell, an induced pluripotent stem cell, an embryonic stem cell (e.g., a human embryonic stem cell), or a human umbilical vein endothelial cell, inter alia. In some embodiments, the epithelial cell is a primary epithelial cell. The seeded matrix can maintained in vitro, e.g., for about 2-14 days or longer.

In another aspect, this document features a functional bioartificial pancreatic tissue provided by the methods provided herein.

In a further aspect, this document features a method of providing insulin-producing cells to a human subject. The method can comprise obtaining a functional bioartificial pancreatic tissue provided by the methods provided herein, and transplanting the tissue into the human subject. One or more of the regenerative cell, the insulin producing cell, and the endothelial cell can be autologous to the subject.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document relates to methods and materials involved in generating functional pancreatic tissues. The present invention is based at least in part on the discovery that cadaveric pancreatic tissue can be decellularized to form a tissue matrix that can be seeded with cells, e.g., human islet cells, to provide functional pancreatic tissue that secretes insulin. For example, seeding a decellularized tissue matrix derived from pancreatic tissue with cells, e.g., human islet cells and stem cells, is associated with substantially increased insulin production, longer survival of islets, and formation of durable endocrine pancreatic tissue. These results suggest a novel method of providing human pancreatic tissues for transplantation. Based at least in part on these discoveries are methods for transplanting a cell-seeded tissue matrix in a human subject to replace or supplement insulin-producing cells.

As used herein, a “functional” pancreatic tissue secretes insulin. A functional pancreatic tissue may secrete insulin in a glucose-sensitive or—insensitive (e.g., constitutive) manner. In some embodiments, the pancreas may also secrete other hormones of the Islet of Langerhans such as glucagon, pancreatic polypeptide, amylin, ghrelin, and somatostatin.

As used herein, the terms “decellularized” and “acellular” are used interchangeably and are defined as the complete or near complete absence of detectable acinar cells, centroacinar cells, ductal cells, islet cells, endothelial cells, smooth muscle cells, and nuclei in histologic sections using standard histological staining procedures. Preferably, but not necessarily, residual cell debris also has been removed from the decellularized organ or tissue.

### Decellularized Tissue/Organ Matrices

Methods and materials for a preparing a composition comprising a decellularized tissue or organ matrix are known in the art. Any appropriate materials can be used to prepare such a composition. In a preferred embodiment, a tissue matrix can be an acellular tissue scaffold developed from any appropriate decellularized tissue. For example, tissue such as human pancreas, or a portion thereof, can be decellularized by an appropriate method to remove native cells from the tissue while maintaining morphological integrity and vasculature of the tissue or tissue portion and preserving extracellular matrix (ECM) proteins. In some cases, cadaveric pancreas or spleen, or portions thereof, can be used. Decellularization methods can include subjecting tissue (e.g., pancreas, spleen, skeletal muscle) to repeated freeze-thaw cycles using liquid nitrogen. In other cases, a tissue can be subjected to an anionic or non-ionic cellular disruption medium such as sodium dodecyl sulfate (SDS), polyethylene glycol (PEG), TRITON®X-100, isopropanol or peracidic acid. The tissue also can be treated with a nuclease solution (e.g., ribonuclease, deoxyribonuclease) and washed in sterile phosphate buffered saline with mild agitation. In some cases, decellularization can be performed by cannulating the vessels, ducts, and/or cavities of the organ or tissue using methods and materials known in the art. Following the cannulating step, the organ or tissue can be perfused via the cannula with a cellular disruption medium as described above. Perfusion through the tissue can be antegrade or retrograde, and directionality can be alternated to improve perfusion efficiency. Depending upon the size and weight of an organ or tissue and the particular anionic or ionic detergent(s) and concentration of anionic or ionic detergent(s) in the cellular disruption medium, a tissue generally is perfused from about 2 to about 12 hours per gram of tissue with cellular disruption medium. Including washes, an organ may be perfused for up to about 12 to about 72 hours per gram of tissue. Perfusion generally is adjusted to physiologic conditions including flow, rate and pressure.

Decellularized tissue can consist essentially of the extracellular matrix (ECM) component of all or most regions of the tissue, including ECM components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. In a preferred embodiment, decellularized pancreatic, splenic, or skeletal muscle matrix retains a substantially intact vasculature. Preserving a substantially intact vasculature enables connection of the tissue matrix to a subject's vascular system upon transplantation. In addition, a decellularized tissue matrix can be further treated with, for example, irradiation (e.g., UV, gamma), or acid (e.g., glacial acetic acid) to reduce or eliminate the presence of any type of microorganism remaining on or in a decellularized tissue matrix.

Methods for obtaining decellularized tissue matrices using physical, chemical, and enzymatic means are known in the art, see, e.g., Liao et al, Biomaterials 29(8):1065-74 (2008); Gilbert et al., Biomaterials 27(9):3675-83 (2006); Teebken et al., Eur. J. Vasc. Endovasc. Surg. 19:381-86 (2000). See also U.S. Pat. Publication Nos. 2009/0142836; 2005/0256588; 2007/0244568; and 2003/0087428.

### Cell Seeding

In the methods described herein, decellularized pancreatic tissue matrix is seeded with cells, e.g., differentiated or regenerative cells. In some embodiments, the cells secrete hormones, e.g., hormones of the Islet of Langerhans such as glucagon, pancreatic polypeptide, amylin, ghrelin, and somatostatin.

Any appropriate regenerative cell type, such as naïve or undifferentiated cell types, can be used to seed the decellularized pancreatic tissue matrix. As used herein, regenerative cells can include, without limitation, progenitor cells, precursor cells, umbilical cord cells (e.g., human umbilical vein endothelial cells (HUVEC)) fetal stem cells, human induced pluripotent stem cells (iPSC), mesenchymal stem cells, multipotent adult progenitor cells (MAPC), or embryonic stem cells. Regenerative cells also can include differentiated or committed cell types. In some cases, regenerative cells derived from other tissues also can be used. For example, regenerative cells derived from skin, bone, muscle, bone marrow, synovium, or adipose tissue can be used to develop stem cell-seeded tissue matrices.

In some cases, a decellularized pancreatic tissue matrix provided herein can be further seeded with differentiated cell types such as human pancreatic islet cells and endothelial cells. For example, a decellularized pancreatic tissue matrix can be seeded with islet cells (e.g., alpha, beta, delta, PP, and/or epsilon cells), mesenchymal cells, and human umbilical vein endothelial cells (HUVEC) through perfusion seeding.

In some embodiments, the decellularized pancreatic tissue matrix can be seeded with insulin-secreting cells and other gastrointestinal hormone secreting cells, e.g., cells that secrete other hormones of the Islet of Langerhans such as glucagon, pancreatic polypeptide, amylin, ghrelin, and somatostatin, derived in vitro from stem or progenitor cells or differentiated cell types. Methods for generating insulin-secreting cells are known in the art, see, e.g., Bonner-Weir et al., PNAS 97:7999-8004 (2000); Ramiya et al., Nat Med 6: 278-282 (2000); Lumelsky et al., Science 292: 1389-1394 (2001); Assady et al., Diabetes 50: 1691-1697 (2001); Gao et al., Diabetes 52: 2007-2015 (2003); Faradji et al., J. Biol. Chem. 276: 36695-36702 (2001); Lipes et al., Acta Diabetologica, 34:2-5 (1997); Kaneto et al., Adv Drug Deliv Rev. 61(7-8):489-96 (2009); and Kaneto et al., J. Biol. Chem. 280 (15): 15047-52 (2005). See also WO00/47720; U.S. Pat. No. 6,001,647; and U.S. Pat. Publication No. 2003/00082810. In some embodiments, the cells secrete insulin in a glucose-sensitive manner. In some embodiments, the cells secrete a basal level of insulin.

Any appropriate method for isolating and collecting cells for seeding can be used. For example, induced pluripotent stem cells generally can be obtained from somatic cells “reprogrammed” to a pluripotent state by the ectopic expression of transcription factors such as Oct4, Sox2, K1f4, c-MYC, Nanog, and Lin28. See Takahashi et al., Cell 131:861-72 (2007); Park et al., Nature 451:141-146 (2008); Yu et al., Science 318:1917-20 (2007). Cord blood stem cells can be isolated from fresh or frozen umbilical cord blood. Mesenchymal stem cells can be isolated from, for example, raw unpurified bone marrow or ficoll-purified bone marrow. Islet cells (e.g., alpha, beta, delta, PP, and/or epsilon cells), and entire Islets of Langerhans can be isolated and collected from living or cadaveric donor pancreases according to methods known in the art. Briefly, proteolytic enzymes perfused through a catheter placed in the pancreatic duct. Portions of the enzymatically treated pancreas can be subjected to further enzymatic and mechanical disruption. The mixture of islet cells and exocrine tissue obtained in this manner can be separated to purify islet cells. In some cases, flow cytometry-based methods (e.g., fluorescence-activated cell sorting) can be used to sort cells based on the presence or absence of specific cell surface markers. In cases where non-autologous cells are used, the selection of immune type-matched cells should be considered, so that the organ or tissue will not be rejected when implanted into a subject. However, the matrix might provide an immuno-privileged site where immune match is not necessary.

Isolated cells can be rinsed in a buffered solution (e.g., phosphate buffered saline) and resuspended in a cell culture medium. Standard cell culture methods can be used to culture and expand the population of cells. Once obtained, the cells can be contacted with a tissue matrix to seed the matrix. For example, a tissue matrix can be seeded with at least one cell type in vitro at any appropriate cell density. For example, cell densities for seeding a matrix can be at least 1×10³ cells/matrix. Cell densities can range between about 1×10³ to about 1×10⁹ cells/matrix (e.g., at least 10,000, 100,000, 1,000,000, 10,000,000, 100,000,000, or 1,000,000,000 cells/matrix) can be used.

In some cases, a decellularized pancreatic tissue matrix as provided herein can be seeded with the cell types and cell densities described above by perfusion seeding. For example, a flow perfusion system can be used to seed the decellularized pancreatic tissue matrix via the vascular system preserved in the tissue matrix. In some cases, automated flow perfusion systems can be used under the appropriate conditions. Such perfusion seeding methods can improve seeding efficiencies and provide more uniform distribution of cells throughout the composition. Quantitative biochemical and image analysis techniques can be used to assess the distribution of seeded cells following either static or perfusion seeding methods.

In some embodiments, e.g., when the matrix is reseeded with whole Islets of Langerhans, or parts of Langerhans organs or islets, the organs or islets might be injected directly into the matrix due to their size.

In some cases, a tissue matrix can be impregnated with one or more growth factors to stimulate differentiation of the seeded regenerative cells. For example, a tissue matrix can be impregnated with connective tissue growth factor (CTGF). CTGF has been shown to be expressed in the pancreas and required for normal islet morphogenesis and embryonic β-cell proliferation. See Crawford et al., Mol. Endocrinol. 23(3):324-336 (2009). Other growth factors appropriate for the methods and materials provided herein can include, for example, vascular endothelial growth factor (VEGF), TGF-β growth factors, bone morphogenetic proteins (e.g., BMP-1, BMP-4), platelet derived growth factor (PDGF), basic fibroblast growth factor (b-FGF), insulin-like growth factor (IGF), epidermal growth factor (EGF), or growth differentiation factor-5 (GDF-5).

Seeded tissue matrices can be incubated for a period of time (e.g., from several hours to about 14 days or more) post-seeding to improve fixation and penetration of the cells in the tissue matrix. The seeded tissue matrix can be maintained under conditions in which at least some of the regenerative cells can multiply and/or differentiate within and on the acellular tissue matrix. Such conditions can include, without limitation, the appropriate temperature and/or pressure, electrical and/or mechanical activity, force, the appropriate amounts of O₂ and/or CO₂, an appropriate amount of humidity, and sterile or near-sterile conditions. In some cases, nutritional supplements (e.g., nutrients and/or a carbon source such as glucose), exogenous hormones, or growth factors can be added to the seeded tissue matrix. Histology and cell staining can be performed to assay for seeded cell propagation. Any appropriate method can be performed to assay for seeded cell differentiation. In some cases, quantitative real-time reverse transcription-polymerase chain reaction (RT-PCR) can be performed to detect and measure expression levels of, for example, markers of differentiated pancreatic beta cells (e.g., Pdx-1, Glut2). In some embodiments, glucose challenge experiments can be performed to detect glucose-sensitive insulin secretion.

Thus the methods described herein can be used to generate a transplantable bioartificial pancreatic tissue, e.g., for transplanting into a human subject. As described herein, a transplantable tissue will preferably retain a sufficiently intact vasculature that can be connected to the patient's vascular system. Alternatively, the tissue can be implanted in certain locations, e.g. into the skeletal muscle, the abdomen, or subcutaneously, and vascularized by capillary ingrowth.

The bioartificial pancreatic tissues described herein can be combined with packaging material to generate articles of manufacture or kits. Components and methods for producing articles of manufacture are well known. In addition to the bioartificial tissues, an article of manufacture or kit can further can include, for example, one or more anti-adhesives, sterile water, pharmaceutical carriers, buffers, and/or other reagents for promoting the development of functional pancreatic tissue in vitro and/or following transplantation. In addition, printed instructions describing how the composition contained therein can be used can be included in such articles of manufacture. The components in an article of manufacture or kit can be packaged in a variety of suitable containers.

### Methods for Using Bioartificial Pancreas Tissue

This document also provides methods and materials for using bioartificial pancreatic tissues and, in some cases, promoting production of insulin and other GI hormones, e.g., other hormones of the Islet of Langerhans such as glucagon, pancreatic polypeptide, amylin, ghrelin, and somatostatin. In some embodiments, the methods provided herein can be used to regenerate a population of insulin-producing islet cells in a human subject in need thereof, e.g., a subject with insulin-dependent diabetes, e.g., Type 1 or Type 2 diabetes. In some embodiments, the methods provided herein can be used to restore some pancreatic activity in patients having diseases that affect the pancreas (e.g., pancreatic cancer, or pancreatitis). The methods provided herein also include those wherein the subject is identified as in need of a particular stated treatment, e.g., increased insulin production.

Bioartificial pancreatic tissues (e.g., whole organs or portions thereof) can be generated according to the methods provided herein. In some embodiments, the methods comprise transplanting a bioartificial pancreatic tissue as provided herein to a subject (e.g., a human patient) in need thereof In some embodiments, a bioartificial pancreatic tissues is transplanted to the site of diseased or damage tissue. For example, bioartificial pancreatic tissues can be transplanted into the abdominal cavity of a subject in place of (or in conjunction with) a non-functioning pancreas; methods for performing pancreatic transplantation are known in the art, see, e.g., Steurer et al., Eur. Surg. 33(1):8-12 (2001); Boggi et al., Transpl. Proc. 37(6):2648-2650 (2005); Tajra et al., Transpl. Intl. 11(4):295-300 (2008); and Cundiff et al., Curr. Surg. 58(2):165-173 (2001)). In another embodiment, the bioartificial pancreas tissue can be implanted heterotopically, e.g. into the groin, subcutaneous tissue of upper and lower extremities and trunk. The methods can include transplanting a bioartificial pancreatic tissue as provided herein during a surgical procedure to partially or completely remove a subject's pancreas and/or during a pancreas resection. In some cases, the methods provided herein can be used to replace or supplement insulin-producing cells in a human subject. For example, a composition described herein can be transplanted into a human subject to replace or supplement insulin-producing islet cells.

Any appropriate method(s) can be performed to assay for cell differentiation, replacement of insulin-producing cells, sustained insulin production, and formation of endocrine pancreatic tissue before or after transplantation. For example, methods can be performed to assess tissue healing, to assess functionality, and to assess cellular in-growth. In some cases, tissue portions can be collected and treated with a fixative such as, for example, neutral buffered formalin. Such tissue portions can be dehydrated, embedded in paraffin, and sectioned with a microtome for histological analysis. Sections can be stained with hematoxylin and eosin (H&E) and then mounted on glass slides for microscopic evaluation of morphology and cellularity. For example, histology and cell staining can be performed to detect seeded cell propagation. Assays can include functional evaluation of the transplanted tissue matrix, analysis of insulin production, or imaging techniques (e.g., computed tomography (CT), ultrasound, magnetic resonance cholangiopancreatography, endoscopic retrograde cholangiopancreatography). Functionality of the matrix seeded with regenerative cells and islet cells can be assayed using methods known in the art, e.g., a glucose response ELISA. The secretion of insulin, glucagon, and other GI hormones, e.g., other hormones of the Islet of Langerhans such as pancreatic polypeptide, amylin, ghrelin, and somatostatin, can be measured as another functionality assay. To assay for islet cell proliferation, islet cell thymidine kinase activity can be measured by, for example, detecting thymidine incorporation. In some cases, blood tests can be performed to evaluate the function of the pancreas based on levels of pancreatic enzymes such as amylase and lipase. In other cases, a secretin stimulation test can be performed to measure the ability of the pancreas to respond to secretin, a hormone made by the small intestine. In other cases, metabolic imaging techniques (e.g., positron emission tomography PET, single photon emission tomography SPECT) can be used to monitor tissue viability and function.

In some cases, molecular biology techniques such as RT-PCR can be used to quantify the expression of metabolic and differentiation markers. For example, RT-PCR and real-time RT-PCR can be used to measure the expression of Pdx-1 (pancreatic and duodenal homeobox 1) and/or Glut2 (glucose transporter 2) which are genetic markers for beta islet cells. Any appropriate RT-PCR protocol can be used. Briefly, total RNA can be collected by homogenizing a biological sample (e.g., pancreas sample), performing a chloroform extraction, and extracting total RNA using a spin column (e.g., RNeasy® Mini spin column (QIAGEN, Valencin, Calif.)) or other nucleic acid-binding substrate. In other cases, markers associated with pancreatic cells types and different stages of differentiation for such cell types can be detected using antibodies and standard immunoassays.

The invention will be further described in the following examples, which do not limit the scope of the invention described in the claims.

### EXAMPLES

### Example—Endocrine Pancreas Matrix Scaffolds

Decellularized pancreas matrix scaffolds were explanted from donor rats using SDS perfusion decellularization through the preserved vascular supply. The matrices were analyzed for integrity of the vascular tree using methylene blue injections. In addition, the matrix was analyzed using standard immunohistochemical methods for the presence of extracellular matrix proteins that have been shown to support islets and Langerhans organ survival. These results demonstrated the presence of Laminin, Fibronectin, Fibrillin, Collagen I, III, IV, X and others.

The matrices were seeded with donor islet cells and supportive mesenchymal stem cells (MSC) via direct delivery. The matrix was endothelialized through perfusion seeding with human umbilical vein endothelial cells (HUVEC). Functionality of the matrix and benefit of supportive MSC to islet organs was proven with glucose response ELISA, as well as Tunnel and Ki67staining.

Pancreas matrix scaffolds were successfully created with intact vascular supply. Seeding with islet cells plus supportive MSC showed superior functional outcome as compared to seeding islet cells alone. An insulin response assay showed preserved insulin response to glucose challenge 2, 5, and 7 days after seeding. Tunnel and Ki67 staining was suggestive of improved resistance to apoptosis active proliferation of islet cells in the matrix seeded with MSC. These data suggest that seeding decellularized pancreas matrices with islet cells, MSC, and HUVEC leads to functional insulin producing scaffolds with superior islet cell survival and function. The matrices were heterotopically transplanted into the abdominal cavity of rats and showed endocrine function in vivo.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for generating a functional bioartificial pancreatic tissue, the method comprising:
providing a decellularized pancreatic tissue matrix comprising vasculature;
directly seeding the decellularized pancreatic tissue matrix with one or both of a hormone secreting cell and a regenerative cell;
seeding the matrix with an endothelial cell by vascular perfusion; and
maintaining the seeded matrix under conditions and for a time sufficient for tissue growth to occur,
thereby generating a functional bioartificial pancreatic tissue.

2. The method of claim 1, further comprising assaying for hormone secretion from the tissue.

3. The method of claim 1, wherein the hormone secreting cells secrete insulin.

4. The method of claim 3, further comprising assaying for insulin secretion from the tissue.

5. The method of claim 1, wherein providing a decellularized pancreatic tissue matrix comprises obtaining tissue comprising a pancreas or a portion thereof comprising vasculature, and decellularizing the pancreas or portion thereof under conditions such that the acellular tissue matrix, including the vasculature, substantially retains morphology of an extracellular matrix of the pancreatic tissue prior to decellularization.

6. The composition of claim 1, wherein the regenerative cell is a mesenchymal stem cell.

7. The composition of claim 1, wherein the regenerative cell is an autologous stem cell.

8. The composition of claim 1, wherein the regenerative cell is an induced pluripotent stem cell or human embryonic stem cell.

9. The composition of claim 1, wherein the endothelial cell is a human umbilical vein endothelial cell.

10. The composition of claim 1, wherein the islet cell secretes a hormone selected from the group consisting of insulin, glucagon, pancreatic polypeptide, amylin, ghrelin, and somatostatin

11. The composition of claim 8, wherein the islet cell secretes insulin or glucagon.

12. The composition of claim 1, wherein the hormone secreting cells is an islet cell.

13. The composition of claim 12, wherein the islet cell is in an intact or partially intact islet of Langerhans.

14. The composition of claim 12, wherein the islet cell is an alpha, beta, delta, PP, or epsilon cell.

15. The composition of claim 1, wherein the seeded matrix is maintained in vitro for 2-14 days or longer.

16. A functional bioartificial pancreatic tissue provided by the method of claim 1.

17. A functional bioartificial pancreatic tissue of claim 16 for the treatment of insulin-dependent diabetes.

18. A method of providing insulin-producing cells to a human subject, the method comprising obtaining a functional bioartificial pancreatic tissue provided by the method of claim 1, and transplanting the tissue into the human subject.

19. The method of claim 18, wherein one or more of the regenerative cell, the insulin producing cell, and the endothelial cell are autologous to the subject.
